(19) United States
(12) Patent Application Publication
(10) Pub. No.: US 2009/0311554 A1
(43) Pub. Date: Dec. 17, 2009

(54) CARBON NANOTUBE DISPERSING AGENT, CARBON NANOTUBE COMPOSITE, CARBON NANOTUBE FILM, AND METHOD FOR MANUFACTURING THE CARBON NANOTUBE FILM

(76) Inventors: Sang Keun Oh, Gyeonggi-do (KR); Kyoung-Hwa Song, Seoul (KR); Da Jeong Jeong, Gyeonggi-do (KR)

Correspondence Address: KED & ASSOCIATES, LLP, P.O. Box 221200, Chantilly, VA, 20153-1200, US

(21) Appl. No.: 12/521,919

(22) PCT Filed: Jan. 4, 2008
(86) PCT No.: PCT/KR2008/000056

§ 371 Date: Jul. 1, 2009

(30) Foreign Application Priority Data
Jan. 5, 2007 (KR) 10-2007-0001623
Jan. 5, 2007 (KR) 10-2007-0001624

(51) Int. Cl. B32B 9/00; C07C 305/04; C07C 43/04; H01B 1/24; B05D 1/02; B05D 5/00; C25D 13/02; B82B 1/00; B82B 3/00

(52) U.S. Cl. 428/688; 558/20; 568/662; 252/510; 427/427; 427/256; 204/471; 977/742; 977/842

(57) ABSTRACT

Provided are a carbon nanotube dispersing agent, a carbon nanotube composite, a carbon nanotube film, and a method for manufacturing the carbon nanotube film. The carbon nanotube dispersing agent has at least one chromophore including at least one aromatic carbon ring, and has a plane structure.

### TECHNICAL FIELD

The present invention relates to a carbon nanotube dispersing agent, a carbon nanotube composite, a carbon nanotube film, and a method for manufacturing the carbon nanotube film, and more particularly, to a carbon nanotube dispersing agent which disperses carbon nanotube fibers with excellent conductivity and dispersivity, a carbon nanotube composite where the carbon nanotube fibers are modified with the carbon nanotube dispersing agent, a carbon nanotube film including the carbon nanotube composite, and a method for manufacturing the carbon nanotube film.

### BACKGROUND ART

Carbon nanotubes (CNTs) have characteristic electrical, chemical properties because carbon atoms are positioned in a hexagonal honeycomb-like pattern to create a tube form. Carbon nanotubes are extremely small materials having a tube diameter in a nanometer size. Carbon nanotubes have superior mechanical properties, electrical selectivity and excellent field emission properties.

Also, since carbon nanotubes have the properties of a semiconductor according to the wound structure and have different energy gaps according to the diameter, carbon nanotubes have been noted in electrical fields, biotechnology fields, medical fields, etc. For example, researches on carbon nanotubes that can be applied to form conductive films and manufacture field emission displays (FEDs) are actively carried out.

Meanwhile, in order to use carbon nanotubes to form conductive films or manufacture various electronic devices, carbon nanotubes have to be effectively dispersed to a matrix such as a binder. However, carbon nanotubes are apt to form bundles in a matrix by a strong Van der Waals force. If carbon nanotubes form bundles in a matrix, the carbon nanotubes may lose their characteristic properties or uniformity may deteriorate when they are manufactured as a thin film.

Current researches into carbon nanotubes have been focused on dispersants for preventing carbon nanotubes from forming bundles, and methods for enhancing or changing the electrical properties of carbon nanotubes.

Also, any research into color carbon nanotubes having various colors has not been carried out. Because various visual products are required along with development of display industries, it is required to manufacture color carbon nanotubes having various colors which can be used in various application fields.

A method of dispersing carbon nanotubes includes a mechanical dispersion method, a dispersion method using a dispersing agent, and a dispersion method using a strong acid. However, since the mechanical dispersion method and the dispersion method using the strong acid can damage carbon nanotubes, the dispersion method using the dispersing agent which can maintain the characteristic properties of carbon nanotubes is generally used. The dispersing agent includes sodium dodecyl sulfate (SDS), Triton X-100, and lithium dodecyl sulfate (LDS), which are surfactants. However, a maximum dispersion density of the dispersing agent is only 1%.

U.S. Pat. No. 6,787,600 discloses a dispersant which comprises a polyamine backbone chain containing side chains of two or more different patterns of polyester chain. Also, U.S. Pat. No. 6,599,973 discloses an aqueous graft copolymer which has a weight average molecular weight of about 5,000-100,000 and comprises a hydrophobic polymeric backbone and discrete anionic and nonionic hydrophilic side chains attached to the backbone.

Also, U.S. Pat. No. 5,530,070 discloses an aqueous metallic flake dispersant formed by polymerizing ethylenically unsaturated monomers, and having macromonomer side chains attached to a polymer backbone.

### DISCLOSURE OF INVENTION

### Technical Problem

However, the above-mentioned dispersants have low solubility and high viscosity because they are polymer dispersants, and accordingly, cannot disperse carbon nanotubes sufficiently. Also, there is a removal problem in a later process because organic solvents are used.

Also, since carbon nanotubes basically include carbon, carbon nanotubes cannot have any other color except for black.

### Technical Solution

The present invention provides a carbon nanotube dispersing agent, having high solubility, low viscosity, and excellent hydrophile property, and having excellent dispersivity even when it is in low concentration.

The present invention also provides a carbon nanotube composite and a carbon nanotube film, having excellent conductivity, and a method for manufacturing the carbon nanotube film, using the carbon nanotube dispersing agent.

The present invention also provides a carbon nanotube composite and a carbon nanotube film, having various colors, and a method for manufacturing the carbon nanotube film.

### ADVANTAGEOUS EFFECTS

According to the present invention, a carbon nanotube film manufactured by uniformly dispersing a large amount of carbon nanotubes in a dispersing solvent has high conductivity without damaging the characteristic properties of carbon nanotubes.

Also, ingredients costs can be reduced, and post-contamination can be prevented compared to other dispersion processes using organic solvents.

Furthermore, since a carbon nanotube composite according to the present invention has a specific color without damaging the characteristic properties of carbon nanotubes, the carbon nanotube composite can be applied to various color products.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 is a cross-section view schematically showing the structure of a carbon nanotube film according to an embodiment of the present invention;

FIG. 2 shows an example of a carbon nanotube composite applied on a substrate for the carbon nanotube film illustrated in FIG. 1;

FIG. 3 is a flowchart of a method for manufacturing a carbon nanotube film, according to an embodiment of the present invention;

FIG. 4 is a graph showing UV spectrums of first and second embodiments of the present invention and first and second comparative examples;

FIG. 5 is a graph showing a UV spectrum of a dye which is applied to the first embodiment of the present invention, and a spectrum when the dye reacts with carbon nanotubes; and

FIG. 6 is a graph for comparing degrees of dispersion of carbon nanotubes in the first embodiment of the present invention to degrees of dispersion of carbon nanotubes in the first and second comparative examples.

### BEST MODE FOR CARRYING OUT THE INVENTION

According to an aspect of the present invention, there is provided a carbon nanotube dispersing agent used to disperse a plurality of carbon nanotube fibers, having at least one chromophore including at least one aromatic carbon ring, and having a plane structure.

The at least one chromophore includes at least one of a nitroso group, a tiocarbonyl group, an ethylene group, an acetylene group, and an azo group, and groups constructing the at least one chromophore are linked by π-electron conjugation.

The carbon nanotube dispersing agent is a dye.

According to another aspect of the present invention, there is provided a carbon nanotube composite, including: a plurality of carbon nanotube fibers contacting each other and dispersed; and a chromophoric compound used to disperse the plurality of carbon nanotube fibers, having at least one chromophore including at least one aromatic carbon ring, and having a plane structure.

According to another aspect of the present invention, there is provided a carbon nanotube film including: a substrate; and a plurality of carbon nanotube composites attached on the substrate and including a plurality of carbon nanotube fibers dispersed by a chromophoric compound, the chromophoric compound having at least one chromophore including at least one aromatic carbon ring, and having a plane structure.

The at least one chromophore includes at least one of a nitroso group, a tiocarbonyl group, an ethylene group, an acetylene group, and an azo group.

The carbon nanotube dispersing agent includes at least two chromophores, and the at least two chromophores are linked together by π-conjugation.

According to another aspect of the present invention, there is provided a carbon nanotube film including: a substrate; and a plurality of carbon nanotube composites attached on the substrate and including a plurality of carbon nanotube fibers dispersed by a chromophoric compound, the chromophoric compound having at least one chromophore including at least one aromatic carbon ring, and having a plane structure.

According to another aspect of the present invention, there is provided a method for manufacturing a carbon nanotube film, including: putting a plurality of carbon nanotube fibers and a carbon nanotube dispersing agent into a dispersing solvent, the carbon nanotube dispersing agent having at least one chromophore including at least one aromatic carbon ring, and having a plane structure; mixing the plurality of carbon nanotube fibers, the carbon nanotube dispersing agent, and the dispersing solvent, to form a carbon nanotube composite; and applying the carbon nanotube composite on a substrate.

It is to be understood that both the foregoing general description and the following detailed description are exemplary and explanatory and are intended to provide further explanation of the invention as claimed.

### MODE FOR THE INVENTION

The invention is described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure is thorough, and will fully convey the scope of the invention to those skilled in the art. In the drawings, the size and relative sizes of layers and regions may be exaggerated for clarity. Like reference numerals in the drawings denote like elements.

A carbon nanotube composite according to an embodiment of the present invention includes a plurality of carbon nanotube (CNT) fibers and a chromophoric compound.

Carbon nanotubes (CNTs) have characteristic electrical, chemical properties because carbon atoms are positioned in a hexagonal honeycomb-like pattern to create a tube form. Carbon nanotubes are extremely small materials having a tube diameter in a nanometer size. Due to the properties, carbon nanotube fibers are apt to form bundles by a strong Van der Waals force. The carbon nanotube fibers are dispersed by a dispersing agent to form a carbon nanotube composite.

The chromophoric compound has at least one chromophore including at least one aromatic carbon ring, and has a plane structure.

The chromophore may be a material selected from among a nitroso group, a tiocarbonyl group, an ethylene group, an acetylene group, an azo group, etc. Also, the chromophore basically includes an aromatic carbon ring in its bidirectional chain. In the current embodiment, the number of chromophores is not limited, and the type of substituent of the aromatic carbon ring is also not limited.

Neighboring chromophores are linked together by π-electron conjugation. Accordingly, since neighboring aromatic carbon rings can form interactions between π-electrons by the chromophores, adsorptive power (π-π interactions) between carbon nanotube dispersing agents and/or between carbon nanotubes and a carbon nanotube dispersing agent becomes excellent.

Also, the chromophoric compound includes an aromatic carbon ring. Hydrocarbons in the aromatic carbon ring can be stably dispersed by separating carbon nanotube fibers lumped by the Van der Waals force through π-stacking interactions with the outer walls of carbon nanotubes. Accordingly, the chromophoric compound can easily disperse the carbon nanotubes without damaging the characteristic properties of carbon nanotubes. Also, the hydrocarbon groups of the chromophores are similar in structure to carbon nanotubes.

Also, the chromophoric compound has a plane structure. Accordingly, the probability that the respective aromatic carbon rings can be coupled with the carbon nanotube fibers is higher than when the chromophoric compound has a structure where aromatic carbon rings are arranged in three-dimension.

The molecule structure of a dye is the same as the molecule structure of the chromophoric compound. Accordingly, the chromophoric compound according to the present invention may be a dye. A dye can be easily purchased and is low in price while having a dispersion effect. Also, since a dye can disperse carbon nanotubes even in a water-soluble solvent, post-contamination can be prevented unlike other dispersion processes using organic solvents.

If a carbon nanotube composite is fabricated using a dye, the carbon nanotube composite has a specific color according to the color of the dye. That is, since the carbon nanotube composite has a specific color without a change in the properties of carbon nanotubes, it can be applied to various color products.

A dye also acts as a dispersing agent. If a dye is comprised of monomers, the dye can have high solubility and low viscosity. Accordingly, since the use of a dye has an advantage capable of dispersing a larger amount of carbon nanotubes than that which a conventional dispersing agent can disperse, a uniform color carbon nanotube composite with excellent dispersivity can be fabricated by adjusting the density of the dye.

Therefore, in the case of dispersing carbon nanotubes using a dye, it is possible to effectively disperse carbon nanotube fibers using the amount which is less than the amount of sodium dodecyl sulfate (SDS), Triton X-100, or lithium dodecyl sulfate (LDS), which is used in a conventional technique. Since the carbon nanotube fibers are dispersed uniformly, the carbon nanotube composite has excellent conductivity and high transmittance when it is formed as a thin film. As a result, since the carbon nanotube composite according to the present invention has excellent conductivity, it can be applied to various electronic, electrical devices requiring electrical properties. Also, the carbon nanotube composite can be used as a conductive film when it is formed as a thin film.

The dye may be a direct dye, an acid dye, a basic dye, a mordant dye, an azoic dye, a sulfur dye, a reactive dye, a disperse dye, etc., which can be commercially purchased or can be made for experimental purposes.

In other words, the dye chemically, structurally includes an azo group, an anthraquinone group, a zanthene group, a triphenylmethane group, a diarylmethane group, a triarylmethane group, a xanthenes group, an indigo group, a phthalocyanine group, etc.

The carbon nanotubes included in the carbon nanotube composite may be single-walled carbon nanotubes, dual-walled carbon nanotubes, multi-walled carbon nanotubes, a bundle of carbon nanotubes, and combinations of the above-mentioned carbon nanotubes. However, the carbon nanotubes which can be applied to the present invention are not limited to the above-mentioned structures.

Meanwhile, the color carbon nanotube composite can further include a polymer resin. In this case, the polymer resin, the carbon nanotube fibers, and the carbon nanotube dispersing agent may have a weight part of 50-99, a weight part of 0.001-30, and a weight part of 0.1-20, respectively, with respect to 100 weight parts of the carbon nanotube composite.

The carbon nanotube fibers included in the carbon nanotube composite may be single-walled carbon nanotube fibers, dual-walled carbon nanotube fibers, multi-walled carbon nanotube fibers, and combinations of the above-mentioned carbon nanotube fibers. However, the carbon nanotube fibers which are applied to the present invention are not limited to the above-mentioned structures.

In this case, the carbon nanotube composite can further include a polymer resin. Here, the polymer resin, the carbon nanotube fibers, and the carbon nanotube dispersing agent may have a weight part of 50-99, a weight part of 0.001-30, and a weight part of 0.1-20, respectively, with respect to 100 weight parts of the carbon nanotube composite.

The carbon nanotube composite according to the present invention has more excellent conductivity and dispersivity than that made using the conventional dispersing agent. The carbon nanotube composite can be applied on a substrate through a simple coating method, and manufactured as a carbon nanotube film. Also, the carbon nanotube composite can be applied to devices such as display panels requiring conductivity and transmittance, as well as to various electronic components.

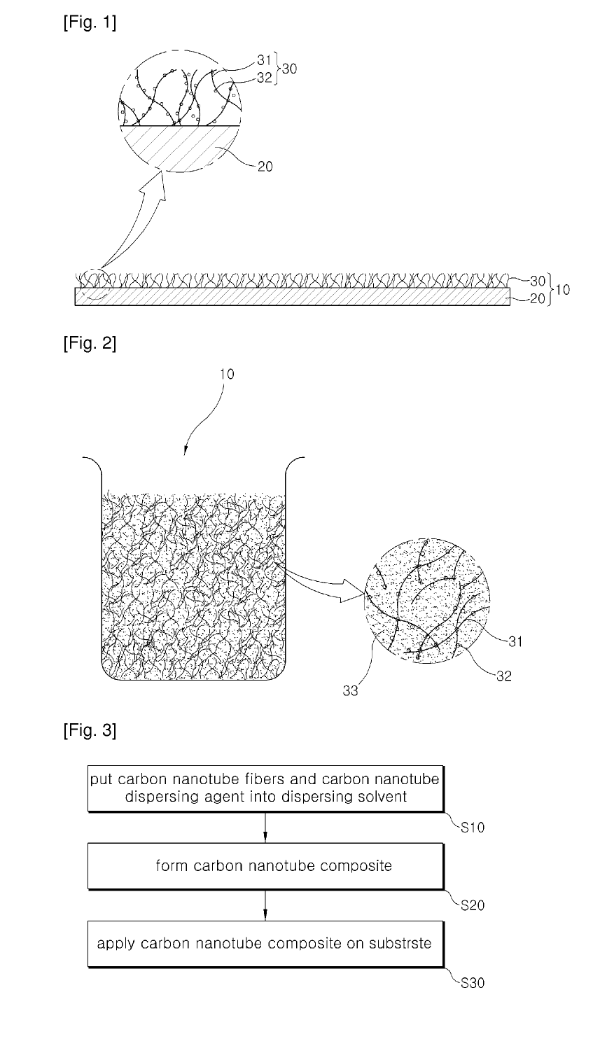

According to an embodiment of the present invention, the carbon nanotube film 10 includes a substrate 20 and a carbon nanotube composite 30, as illustrated in FIG. 1. In this case, the carbon nanotube composite 30 is attached onto the substrate 20, and includes a plurality of carbon nanotube fibers 31. The carbon nanotube fibers are dispersed by a carbon nanotube dispersing agent 32 which has at least one chromophore including at least one aromatic carbon ring and has a plane structure.

The carbon nanotube composite 30 can be coated on the substrate 20, using one of coating methods, such as spraying, spin-coating, electrophoresis deposition, casting, inkjet printing, and offset printing.

As illustrated in FIG. 2, the carbon nanotube composite 30 can further include a dispersing solvent 33. The carbon nanotube fibers 30 and the carbon nanotube dispersing agent 32 are mixed in the dispersing solvent 33. In this case, the dispersing solvent 33, the carbon nanotube fibers 31, and the carbon nanotube dispersing agent 32 may have a weight part of 70-99, a weight part of 0.001-20, and a weight part of 0.01-10, respectively. If the amount of the dispersing solvent is less than the above-mentioned amount, dispersion cannot effectively occur, and, if the amount of the dispersing solvent is more than the above-mentioned amount, the dispersing solvent influences the properties of the film because the dispersing solvent remains.

After the carbon nanotube composite 30 is applied on the substrate 20, all or some of the carbon nanotube dispersing agent 32 included in the carbon nanotube composite 30 can be removed by volatilization, washing, decomposition, etc., after a carbon nanotube film is manufactured.

FIG. 3 is a flowchart of a method for manufacturing a carbon nanotube film, according to an embodiment of the present invention.

Referring to FIG. 3, the method for manufacturing the carbon nanotube film includes operation S10 of putting a plurality of carbon nanotube fibers and a carbon nanotube dispersing agent into a dispersing solvent, operation S20 of mixing the carbon nanotube fibers, the carbon nanotube dispersing agent, and the dispersing solvent to form a carbon nanotube composite, and operation S30 of applying the carbon nanotube composite on a substrate.

First, the plurality of carbon nanotube fibers and the carbon nanotube dispersing agent are put into the dispersing solvent. Here, the carbon nanotube dispersing agent, which has a plane structure, has at least one chromophore including at least one aromatic carbon ring. The carbon nanotube dispersing agent may be a dye.

The chromophore may be a material selected from among a nitroso group, a tiocarbonyl group, an ethylene group, an acetylene group, an azo group, etc. Also, the chromophore basically includes an aromatic carbon ring in its bidirectional chain. In the current embodiment, the number of chromophores is not limited, and the type of substituent of the aromatic carbon ring is also not limited.

Neighboring chromophores are linked together by π-electron conjugation. Accordingly, since neighboring aromatic carbon rings can form interactions between π-electrons by the chromophores, adsorptive power (π-π interactions) between carbon nanotube dispersing agents and/or between carbon nanotubes and a carbon nanotube dispersing agent becomes excellent.

Also, the chromophore includes an aromatic carbon ring. Hydrocarbons in the aromatic carbon ring can be stably dispersed by separating carbon nanotube fibers lumped by the Van der Waals force through π-stacking interactions with the outer walls of carbon nanotubes. Accordingly, the dispersing agent can easily disperse the carbon nanotubes without damaging the characteristic properties of carbon nanotubes. Also, the aromatic hydrocarbon groups of the dispensing agent are similar in structure to carbon nanotubes.

Also, the carbon nanotube dispensing agent has a plane structure. Accordingly, the probability that the respective aromatic carbon rings can be coupled with the carbon nanotube fibers is higher than when the dispersing agent has a structure where aromatic carbon rings are arranged in three-dimension.

Therefore, in the case of dispersing the carbon nanotubes using the carbon nanotube dispersing agent, it is possible to effectively disperse the carbon nanotube fibers using the amount which is less than that of sodium dodecyl sulfate (SDS), Triton X-100, or lithium dodecyl sulfate (LDS), which is used in the conventional technique.

The carbon nanotube dispersing agent may be a dye. A dye can be easily purchased and is low in price while having a dispersion effect. Also, since a dye can disperse carbon nanotubes in a water-soluble solvent, post-contamination can be prevented unlike other dispersion processes using organic solvents.

The dye may be a direct dye, an acid dye, a basic dye, a mordant dye, an azoic dye, a sulfur dye, a reactive dye, a disperse dye, etc., which can be commercially purchased or can be made for experimental purposes.

In other words, the dye chemically, structurally includes an azo group, an anthraquinone group, a zanthene group, a triphenylmethane group, a diarylmethane group, a triarylmethane group, an xanthenes group, an indigo group, a phthalocyanine group, etc.

The dispersing solvent may be water, alcohols such as methanol and ethanol, ketones, ethers, etc. However, the dispersing solvent is not limited to the above-mentioned materials, and polyvinyle alcohol (PVA), polyacylamide (PAM), and polyacrylic acid polymer can be used as a dispersing matrix.

In this case, the carbon nanotube fibers, the dye, and the dispensing solvent may have a weight part of 0.001-20, a weight part of 0.01-10, and a weight part of 70-99, re-spectively. If the amount of the dispersing solvent is less than the above-mentioned amount, dispersion cannot effectively occur, and if the amount of the dispersing solvent is more than the above-mentioned amount, the dispersing solvent influences the properties of the film because the dispersing solvent remains.

The conductivity of a carbon nanotube film is influenced directly by uniformity in distribution of carbon nanotubes in the carbon nanotube film, and by the density of the dispersing agent. Since the carbon nanotube dispersing agent according to the present invention has an advantage capable of dispersing a larger amount of carbon nanotube fibers than that which the conventional dispersing agent can disperse, a uniform carbon nanotube composite with excellent dispersivity can be manufactured by adjusting the density of the dispersing agent.

Thereafter, the carbon nanotube fibers, the dye, and the dispersing solvent are mixed to form a carbon nanotube composite. At this time, a stirring apparatus, such as a homogenizer, a spiral mixer, a planetary mixer, a disperser, and a hybrid mixer, can be used.

The operation can further include operation of dividing the carbon nanotube composite into a carbon nanotube composite containing carbon nanotube fibers with uniform particles, and a carbon nanotube composite containing carbon nanotube fibers with relatively non-uniform particles. In this operation, the carbon nanotube composite is centrifugally rotated using a centrifugal machine, and the carbon nanotube composite containing the carbon nanotube fibers with relatively uniform particles is extracted from the upper layer of the carbon nanotube composite centrifugally rotated.

Then, the extracted carbon nanotube composite is applied on the substrate. A method of applying the carbon nanotube composite on the substrate may be one of coating methods, such as spraying, spin-coating, electrophoresis deposition, casting, inkjet printing, and offset printing.

The substrate may be glass, a polymer film, membrane, etc. The carbon nanotube composite can be uniformly applied on a flat substrate.

Hereinafter, embodiments of the present invention will be described in more detail.

### First Comparative Example

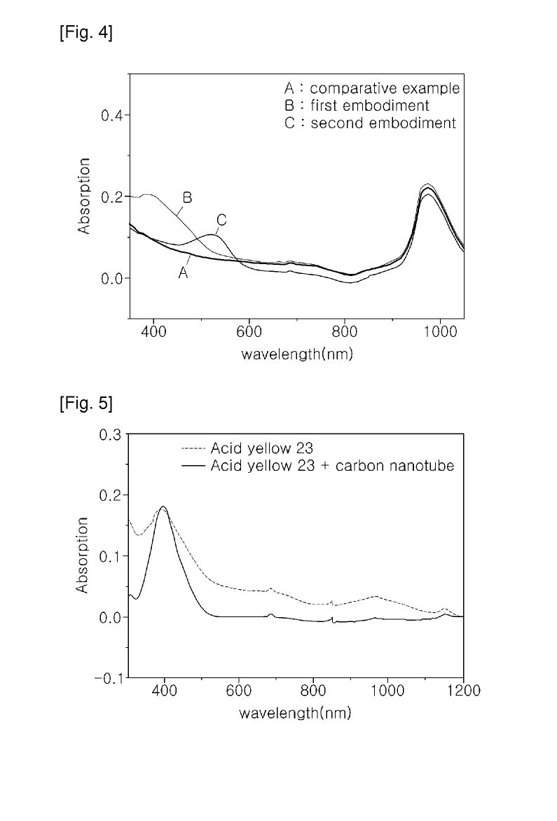

Sodium dodecyle sulfate (SDS) of 2000 mg is used as a dispersing agent in a dispersing solvent. The dispersing solvent is distilled water. In order to manufacture a carbon nanotube film, single-walled carbon nanotubes of 3.0 mg and a dispersing agent of 2000 mg are stirred into distilled water of 200 ml, and they are sufficiently mixed with each other. Then, the carbon nanotubes are dispersed for one hour using a bath sonicator (Branson5510 40 kHz 135 W). The result is measured by UV-Vis-spectroscopy, and a spectrum denoted by a curve A of FIG. 4 is obtained.

After centrifugally rotating the carbon nanotube dispersed solution dispersed by the centrifugal machine for one hour at 6000 rpm, the upper layer of the resultant solution are extracted and the extracted layer is used as a carbon nanotube layer. Then, the carbon nanotube layer is sprayed on a glass substrate plate which is placed on a heating plate, using a spraying method, thereby forming a carbon nanotube film.

Then, the transmittance and sheet resistance of the carbon nanotube film manufactured by the above-described method are measured, respectively, using a turbidimeter (NIPPON DENSHOKU NDH2000) and an electrometer (Loresta-EP MCP-T360) with 4-point probes based on ASTM D257.

In the first comparative example, the transmittance and sheet resistance of the carbon nanotube film are respectively measured as 533.8 Ω/sq and 78.2%, as shown in Tables 1 and 2.

### Second Comparative Example

In the second comparative example, Triton X-100 (TX-100) of 1500 mg is used as a dispersing agent in a dispersing solvent. A carbon nanotube film is manufactured under the same condition as in the first comparative example, except for the type and dose of the dispersing agent. Then, the transmittance and sheet resistance of the carbon nanotube film are measured, respectively, using a turbidimeter (NIPPON DENSHOKU NDH2000) and an electrometer (Loresta-EP MCP-T360) with 4-point probes based on ASTM D257.

In the second comparative example, the transmittance and sheet resistance of the carbon nanotube film are respectively measured as 530.3 Ω/sq and 78%, as shown in Tables 1 and 2.

### First Embodiment

A color carbon nanotube composite with a yellow color is manufactured. In the first embodiment, Acid Yellow 23 of 1.5 mg is used as a chromophoric compound, and no separate dispersing agent is used.

A carbon nanotube composite and a carbon nanotube film are manufactured under the same condition as in the first comparative example, except of the type and dose of the dispersing agent.

The carbon nanotube composite has a spectrum denoted by a curve B of FIG. 4 when it is measured by UV-Vis-spectroscopy. At a wavelength smaller than 500 nm, the absorption rate of the first embodiment is greater than those of the first and second comparative examples, and accordingly, the carbon nanotube composite has a yellow color.

FIG. 5 is a graph showing the spectrum of the Acid Yellow 23 which is applied to the first embodiment of the present invention, and a change in the spectrum of the Acid Yellow 23 when the Acid Yellow 23 reacts with carbon nanotube fibers. The change in the spectrum of the Acid Yellow 23 is made because the electronic structures of the Acid Yellow 23 and carbon nanotubes have changed due to interactions when the Acid Yellow 24 is absorbed into the carbon nanotube fibers.

Meanwhile, the transmittance and sheet resistance of a carbon nanotube film which is manufactured using the carbon nanotube composite as a major material are measured, respectively, using the turbidimeter (NIPPON DENSHOKU NDH2000) and the electrometer (Loresta-EP MCP-T360) with 4-point probes based on ASTM D257.

As the result of the measurement, the transmittance of the carbon nanotube film according to the first embodiment is 83.2%, which is significantly greater than those of the first and second comparative examples, at sheet resistance of 577.9 Ω/sq which is similar to those of the first and second comparative examples, as shown in Table 1. Also, when the transmittance of the carbon nanotube film is 77.2% which is similar to those of the first and second comparative examples, as shown in Table 2, the sheet resistance of the carbon nanotube film is 254.8 Ω/sq which is significantly smaller than those of the first and second comparative examples. That is, the carbon nanotube film according to the first embodiment has more excellent transmittance at the same resistance than those of the first and second comparative examples, and has significantly lower resistance at the same transmittance than those of the first and second comparative examples. That is, the carbon nanotube film according to the first embodiment is excellent in transmittance and electrical conductivity.

Comparing the first embodiment to the first and second comparative examples, excellent transmittance and low sheet resistance can be obtained by using only the amount of a dispersing agent which corresponds to about 1/1000 of the amount (2000 mg) of the dispersing agent used in the first comparative example and the amount (1500 mg) of the dispersing agent used in the second comparative example.

Also, in order to compare a dispersing effect of the first embodiment to those of the first and second comparative examples, the transmittance of the carbon nanotube composite is measured. A carbon nanotube composite in which carbon nanotubes are uniformly dispersed will have low transmittance, and a carbon nanotube composite in which carbon nanotubes are non-uniformly dispersed will have high transmittance. This is because the particles of a carbon nanotube composite uniformly dispersed are not deposited and are in a stable state although a constant time elapses, but the particles of a carbon nanotube composite non-uniformly dispersed are deposited with the elapse of time.

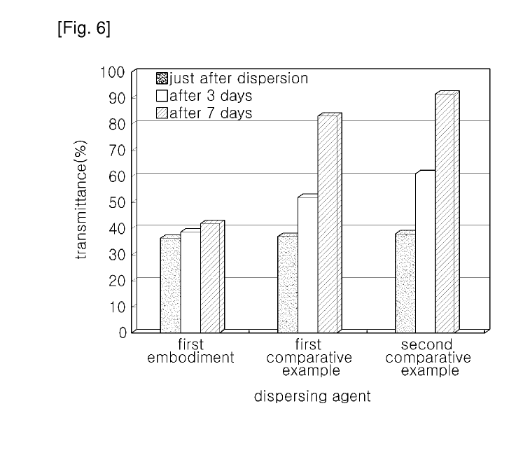

As shown in FIG. 6, in the case of the first embodiment, the transmittance of a carbon nanotube-dispersed solution is little changed between when carbon nanotubes are just dispersed, when three days elapse after carbon nanotubes are dispersed, and when seven days elapse after carbon nanotubes are dispersed. Meanwhile, in the cases of the first and second comparative examples, the transmittance of a carbon nanotube-dispersed solution has increased two or more times that of the first embodiment when seven days elapse after carbon nanotubes are dispersed. Therefore, the first embodiment in which a dye is used as a dispersing agent is excellent in dispersivity compared to the first and second comparative examples in which normal dispersing agents are used.

### Second Embodiment

### Basic Blue 41 is Used as a Dispersing Agent

In the second embodiment, Basic Blue 41 of 1.5 mg is used as a dispersing agent, and put into a dispersing solvent. A carbon nanotube film is manufactured under the same condition as in the first embodiment, except for the type and dose of the dispersing agent. Then, the transmittance and sheet resistance of the carbon nanotube film are measured, respectively, using the turbidimeter (NIPPON DENSHOKU NDH2000) and the electrometer (Loresta-EP MCP-T360) with 4-point probes based on ASTM D257.

As the result of the measurement, the transmittance of the carbon nanotube film according to the second embodiment is 81.8%, which is significantly greater than those of the first and second comparative examples, at sheet resistance of 599.4 Ω/sq which is similar to those of the first and second comparative examples, as shown in Table 1. Also, when the transmittance of the carbon nanotube film is 74% which is similar to those of the first and second comparative examples, as shown in Table 2, the sheet resistance of the carbon nanotube film is 317 Ω/sq which is significantly smaller than those of the first and second comparative examples. That is, the carbon nanotube film according to the second embodiment has more excellent transmittance at the same sheet resistance than those of the first and second comparative examples, and has significantly lower resistance at the same transmittance than those of the first and second comparative examples. That is, the carbon nanotube film according to the second embodiment is excellent in transmittance and electrical conductivity.

Comparing the second embodiment to the first and second comparative examples, excellent transmittance and low sheet resistance can be obtained by using only the amount of a dispersing agent which corresponds to about 1/1000 of those used in the first and second comparative examples.

### Third Embodiment

A color carbon nanotube composite with a red color is manufactured. In the current embodiment, Acid Red 88 of 1.5 mg is used as a chromophoric compound, and no separate dispersing agent is used.

A carbon nanotube composite and a carbon nanotube film are manufactured under the same condition as in the first embodiment, except for the type and dose of the chromophoric compound.

The carbon nanotube composite has a spectrum denoted by a curve C of FIG. 4 when it is measured by UV-Vis-spectroscopy. At a wavelength from 500 nm to 600 nm, the absorption rate of the second embodiment is greater than those of the first and second comparative examples, and accordingly, the carbon nanotube composite has a red color.

The transmittance and sheet resistance of the carbon nanotube film are measured, respectively, using the turbidimeter (NIPPON DENSHOKU NDH2000) and the electrometer (Loresta-EP MCP-T360) with 4-point probes based on ASTM D257.

As the result of the measurement, the transmittance of the carbon nanotube film according to the third embodiment is 81.8%, which is significantly greater than those of the first and second comparative examples, at sheet resistance of 552.0 Ω/sq which is similar to those of the first and second comparative examples, as shown in Table 1. Also, when the transmittance of the carbon nanotube film is 76.2% which is similar to those of the first and second comparative examples, as shown in Table 2, the sheet resistance of the carbon nanotube film is 329 Ω/sq which is significantly smaller than those of the first and second comparative examples. That is, the carbon nanotube film according to the third embodiment has more excellent transmittance at the same sheet resistance than those of the first and second comparative examples, and has significantly lower resistance at the same transmittance than those of the first and second comparative examples. That is, the carbon nanotube film according to the third embodiment is excellent in transmittance and electrical conductivity.

Also, compared the third embodiment to the first and second comparative examples, excellent transmittance and low sheet resistance can be obtained by using only the amount of a dispersing agent which corresponds to about 1/1000 of those used in the first and second comparative examples.

TABLE 1

| | Sheet resistance | |
|---|---|---|
| | (Ω/sq) | Transmittance (%) |
| First embodiment | 577.9 | 83.2 |
| Second embodiment | 599.4 | 81.8 |
| Third embodiment | 552.0 | 82.0 |
| First comparative example | 533.8 | 78.2 |
| Second comparative example | 530.3 | 78.0 |

TABLE 2

| | Sheet resistance | |
|---|---|---|
| | (Ω/sq) | Transmittance (%) |
| First embodiment | 254.8 | 77.2 |
| Second embodiment | 317.0 | 74.0 |
| Third embodiment | 329.0 | 73.2 |
| First comparative example | 533.8 | 78.2 |
| Second comparative example | 530.3 | 78.0 |

According to the present invention, by using the above-described dispersing agent, a thin carbon nanotube film in which a large amount of carbon nanotubes is uniformly dispersed in a dispersing solvent can be manufactured so that it has high conductivity without damaging the properties of carbon nanotubes.

Also, since a dye which can be easily purchased and is low in price can be used as a dispersing agent, ingredient costs can be reduced. Also, since carbon nanotubes can be dispersed in a water-soluble solvent, post-contamination can be prevented unlike other dispersion processes using organic solvents.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

### INDUSTRIAL APPLICABILITY

The present invention can be applied to electronics industry, biotechnology fields, medical industry, etc.

1. A carbon nanotube dispersing agent used to disperse a plurality of carbon nanotube fibers, having at least one chromophore including at least one aromatic carbon ring, and having a plane structure.

2. The carbon nanotube dispersing agent of claim 1, wherein the at least one chromophore comprises at least one of a nitroso group, a tiocarbonyl group, an ethylene group, an acetylene group, and an azo group, and groups constructing the at least one chromophore are linked by π-electron conjugation.

3. The carbon nanotube dispersing agent of claim 1 being a dye.

4. The carbon nanotube dispersing agent of claim 3, wherein the dye is any one selected from among a direct dye, an acid dye, a basic dye, a mordant dye, an azoic dye, a sulfur dye, a reactive dye, and a disperse dye.

5. A carbon nanotube composite, comprising:
a plurality of carbon nanotube fibers contacting each other and dispersed; and
a chromophoric compound used to disperse the plurality of carbon nanotube fibers, having at least one chromophore including at least one aromatic carbon ring, and having a plane structure.

6. The carbon nanotube composite of claim 5, wherein the at least one chromophore comprises at least one of a nitroso group, a tiocarbonyl group, an ethylene group, an acetylene group, and an azo group.

7. The carbon nanotube composite of claim 6, wherein groups constructing the at least one chromophore are linked by π-electron conjugation.

8. The carbon nanotube composite of claim 5, wherein the chromophoric compound is a dye.

9. The carbon nanotube composite of claim 8, wherein the dye is any one selected from among a direct dye, an acid dye, a basic dye, a mordant dye, an azoic dye, a sulfur dye, a reactive dye, and a disperse dye.

10. The carbon nanotube composite of claim 5, further comprising a polymer resin, wherein
the polymer resin, the plurality of carbon nanotube fibers, and the carbon nanotube dispersing agent have a weight part of 50-99, a weight part of 0.001-30, and a weight part of 0.1-20, respectively, with respect to 100 weight parts of the carbon nanotube composite.

11. The carbon nanotube composite of claim 5, wherein the chromophoric compound is a dye, and the carbon nanotube composite has a specific color.

12. A carbon nanotube film comprising:
substrate; and
a plurality of carbon nanotube composites attached on the substrate and including a plurality of carbon nanotube fibers dispersed by a chromophoric compound, the chromophoric compound having at least one chromophore including at least one aromatic carbon ring, and having a plane structure.

13. The carbon nanotube film of claim 12, wherein the at least one chromophore comprises at least one of a nitroso group, a tiocarbonyl group, an ethylene group, an acetylene group, and an azo group.

14. The carbon nanotube film of claim 13, wherein groups constructing the at least one chromophore are linked by π-electron conjugation.

15. The carbon nanotube film of claim 12, wherein the chromophoric compound is a dye.

16. The carbon nanotube film of claim 15, wherein the dye is any one selected from among a direct dye, an acid dye, a basic dye, a mordant dye, an azoic dye, a sulfur dye, a reactive dye, and a disperse dye.

17. A method for manufacturing a carbon nanotube film, comprising:
putting a plurality of carbon nanotube fibers and a carbon nanotube dispersing agent into a dispersing solvent, the carbon nanotube dispersing agent having at least one chromophore including at least one aromatic carbon ring, and having a plane structure;
mixing the plurality of carbon nanotube fibers, the carbon nanotube dispersing agent, and the dispersing solvent, to form a carbon nanotube composite; and
applying the carbon nanotube composite on a substrate.

18. The method of claim 17, wherein the carbon nanotube dispersing agent is a dye selected from among a direct dye, an acid dye, a basic dye, a mordant dye, an azoic dye, a sulfur dye, a reactive dye, and a disperse dye.

19. The method of claim 17, wherein, in the putting of the plurality of carbon nanotube fibers and the carbon nanotube dispersing agent into the dispersing solvent, the dispersing solvent, the plurality of carbon nanotube fibers, and the carbon nanotube dispersing agent have a weight part of 70-99, a weight part of 0.001-20, and a weight part of 0.01-10, respectively.

20. The method of claim 17, wherein the dispersing solvent is any one selected from among water, alcohols, ketones, ethers, and polymer matrixes.
